(19) United States
(12) Patent Application Publication
(10) Pub. No.: US 2010/0125201 A1
(43) Pub. Date: May 20, 2010

(54) ULTRASOUND IMAGING APPARATUS

(75) Inventors: Tomokazu FUJII, Nasushiobara-shi (JP); Kazutoshi Sadamitsu, Otawara-shi (JP); Yutaka Kobayashi, Nasushiobara-shi (JP); Satoshi Matsunaga, Nasushiobara-shi (JP); Jiro Higuchi, Otawara-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 12/615,652

(22) Filed: Nov. 10, 2009

(30) Foreign Application Priority Data
Nov. 18, 2008 (JP) 2008-294968

(51) Int. Cl. A61B 8/14

(52) U.S. Cl. 600/443

(57) ABSTRACT

Ultrasound is sent and reflected waves are received, and an image is generated based on the reflected waves. Then, each umbilical cord present in the image is respectively specified, and the respective structures continuing into one end of the specified umbilical cords are specified. Each specified structure is identified and displayed on a monitor by color separation, screen division, display switching, etc.

### BACKGROUND OF THE INVENTION

1. Field of the Invention

The present invention relates to an ultrasound imaging apparatus that generates an image by transmitting and receiving ultrasound. More particularly, the present invention relates to an ultrasound imaging apparatus that supports identifying multiple fetuses present in the womb.

2. Description of the Related Art

An ultrasound imaging apparatus is an apparatus that generates an image by transmitting and receiving ultrasound to and from the inside of a subject. An examination using this ultrasound imaging apparatus is less invasive for the subject. Therefore, this examination can be repeatedly performed on the subject. Also, with an ultrasound imaging apparatus, it is easy to implement a real time display. For these reasons, an ultrasound imaging apparatus is often utilized in the field of obstetrics when tracking a fetus present in the womb of a pregnant woman over time (e.g., see Japanese published unexamined application No. 2001-120544). Recently, a three-dimensional technology for an ultrasound imaging apparatus has been developed.

Physicians and others may confirm the condition of the fetus in three dimensions based on this three-dimensional technology in order to comprehend the development and shape of the fetus.

In the tracking of the fetus, physicians and others determine whether the fetus is growing well or not by representing its development using a growth curve. When multiple fetuses are present in the womb, these growth curves are used to represent each fetus.

Therefore, in order to confirm the degree of growth for each of the multiple fetuses, physicians and others must specify the same fetuses in both the previous and current examinations by distinguishing the fetuses in the womb based on their individual experiences.

However, in ultrasonic images, it is rare that multiple fetuses are clearly distinguished and represented. Thus, it has not been easy to specify each of the multiple fetuses. Therefore, physicians and others might make a mistake in specifying each of multiple fetuses.

### SUMMARY OF THE INVENTION

The present invention is considered in the light of the aforementioned background. The object of the present invention is to provide an ultrasound imaging apparatus that supports physicians and others in specifying the same fetuses when multiple fetuses are present in the womb.

The first aspect of this invention is an ultrasound imaging apparatus having a probe configured to transmit ultrasound and receive the reflected waves, a generator configured to generate an image based on the reflected waves, and a display that displays the image, wherein the ultrasound imaging apparatus comprises: a first analyzer configured to specify respectively each umbilical cord present in said image; a second analyzer configured to respectively specify a structure continuing into one end of said umbilical cord; and a display controller configure to cause each said structure to be displayed on said display.

According to the first embodiment of this invention, even when multiple fetuses are present in the womb, it will be easy to confirm the condition of each fetus by physicians and others, and possible to improve the accuracy of measuring the growth condition of the fetuses, etc.

### BRIEF DESCRIPTION OF THE DRAWINGS

FIG. 1 is a configuration diagram showing the appearance of an ultrasound imaging apparatus according to the present embodiment.

FIG. 2 is a configuration diagram showing an ultrasound probe.

FIG. 3 is a configuration diagram showing an internal configuration of an apparatus body.

FIG. 4 is a diagram showing a detailed configuration of an analyzer according to the first embodiment.

FIG. 5 is a pattern diagram showing a Color Doppler image of an umbilical cord.

FIG. 6 is a pattern diagram showing a B-mode image of a region including a fetus.

FIG. 7 is a flowchart showing a process of specifying an umbilical cord.

FIG. 8 is a flowchart showing a process of specifying a structure.

FIG. 9 is a flowchart showing a process of identifying and displaying a structure.

FIG. 10 is a pattern diagram showing a screen from the ultrasound imaging apparatus.

FIG. 11 is a pattern diagram showing a second aspect of a screen from the ultrasound imaging apparatus.

FIG. 12 is a pattern diagram showing a third aspect of a screen from the ultrasound imaging apparatus.

FIG. 13 is a configuration diagram of an analyzer according to the second embodiment.

FIG. 14 is a data configuration diagram for data including placenta positional information.

FIG. 15 is a pattern diagram showing a display of a probe movement route.

FIG. 16 is a flowchart showing an operation for storing the placenta position, etc.

FIG. 17 is a flowchart showing an operation for reproducing an aspect of identifying the fetus, even for a new examination.

### DETAILED DESCRIPTION OF THE PREFERRED EMBODIMENTS

A preferred embodiment of an ultrasound imaging apparatus according to the present invention is specifically described as follows, with reference to the drawings.

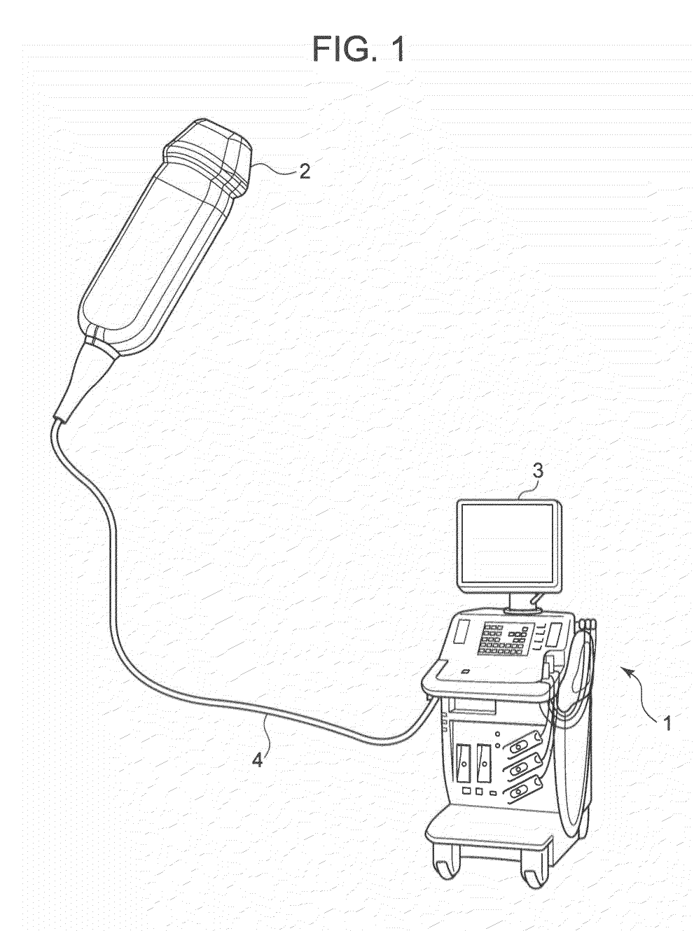

FIG. 1 is a configuration diagram showing the appearance of the ultrasound imaging apparatus according to the present embodiment.

The ultrasound imaging apparatus 1 transmits and receives ultrasound to and from the inside of a subject such as a pregnant woman. Moreover, the ultrasound imaging apparatus 1 generates and displays an image of the inside of the subject based on reflected waves that have been received. More particularly, if tracking of fetuses is an item that is part of the examination, this ultrasound imaging apparatus 1 identifies and displays multiple fetuses in the image.

This ultrasound imaging apparatus 1 is configured to include an ultrasound probe 2 and an apparatus body 3. The ultrasound probe 2 and the apparatus body 3 are connected via a cable 4.

The ultrasound probe 2 is controlled by the apparatus body 3 and transmits and receives ultrasound to and from the inside of the subject. The apparatus body 3 generates ultrasonic images such as B-mode (Brightness Mode) and Color Doppler mode images by processing signals of the reflected waves that have been received. A B-mode image represents the intensity of the received ultrasound using brightness, and represents each position of those tissues in the subject that reflect the ultrasound sent from ultrasound probe 2, arranged two-dimensionally or three-dimensionally. A Color Doppler mode image is an image of blood flow information, the image representing frequency changes in the ultrasound, converted into the flow rate of blood flow using colors. In general, in a Color Doppler mode image, something that is close to the ultrasound probe 2 is processed using red, and something that is distant using blue.

When physicians and others implement three-dimensional scanning with the ultrasound imaging apparatus 1, the physicians and others move the ultrasound probe 2 held by them in a direction perpendicular to the scan plane. Three-dimensional scanning is scanning in which a scan frame (scan plane) is manually or mechanically moved in a three-dimensional region of the subject to collect a signal received from each point within that three-dimensional region.

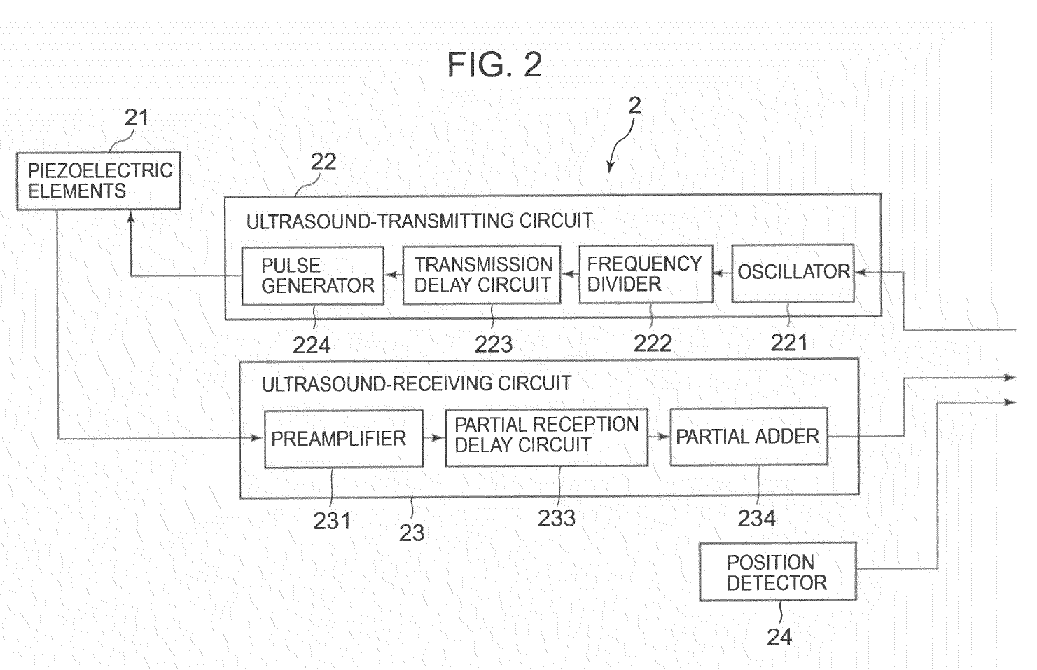

FIG. 2 is a configuration diagram showing the ultrasound probe 2.

The ultrasound probe 2 comprises multiple aligned piezoelectric elements 21, an ultrasound-transmitting circuit 22, an ultrasound-receiving circuit 23, and a position detector 24.

The piezoelectric element 21 is an acoustically/electrically reversible conversion element consisting of a piezoceramic such as lead titanate. When a signal voltage is applied to the piezoelectric element 21, the signal voltage produces a piezoelectric effect, and ultrasound is sent from the piezoelectric element 21. Also, when the piezoelectric element 21 receives ultrasound reflected from the subject, a piezoelectric effect is produced by the ultrasound. Ultrasound is converted by the piezoelectric effect into an echo signal having a voltage value according to the intensity of that ultrasound.

The ultrasound-transmitting circuit 22 applies a voltage pulse to each piezoelectric element 21. This ultrasound-transmitting circuit 22 has an oscillator 221, a frequency divider 222, a transmission delay circuit 223, and a pulse generator 224.

The oscillator 221 controls the transmission timing of voltage pulses. The oscillator 221 generates a timing signal for each predefined time period, and transmits the timing signal to the transmission delay circuit 223 via the frequency divider 222. The frequency divider 222 reduces the timing signal to a rate pulse of, e.g., about 5 KHz.

The transmission delay circuit 223 determines the scanning direction of the ultrasound beam. The transmission delay circuit 223 delays the timing signal per piezoelectric element 21, setting a time lag.

This transmission delay circuit 223 receives a scanning direction signal that selects the scanning direction from the apparatus body 3.

Moreover, the transmission delay circuit 223 sets a delay in applying voltage pulse to each piezoelectric element 21 so that the ultrasound beam is focused in the scanning direction based on the received signal.

The pulse generator 224 applies a voltage pulse to each piezoelectric element 21 in tune with the timing signal received from the transmission delay circuit 223.

The ultrasound-receiving circuit 23 receives an echo signal output by each piezoelectric element 21. This ultrasound-receiving circuit 23 has a preamplifier 231, a partial reception delay circuit 233, and a partial adder 234.

The preamplifier 231 amplifies echo signals. The partial reception delay circuit 233 provides a delay time that is required to determine the receiving directionality for the amplified echo signals.

In order to decrease the number of signal lines in the cable 4, once the partial adder 234 partially phases and adds signals from each piezoelectric element 21, it decreases the number of signal lines to, e.g., 16. The partial adder 234 sends the partially phased and added echo signals respectively via the cable 4 to the apparatus body 3.

The position detector 24 detects positional information indicating the position of the ultrasound probe 2. The positional information is information indicating the reference position PO (x0, y0, z0), angle θa (rotational angle around the X-axis), and angle θB (rotational angle around the Y-axis), angle θY (rotational angle around the Z-axis) of the ultrasound probe 2.

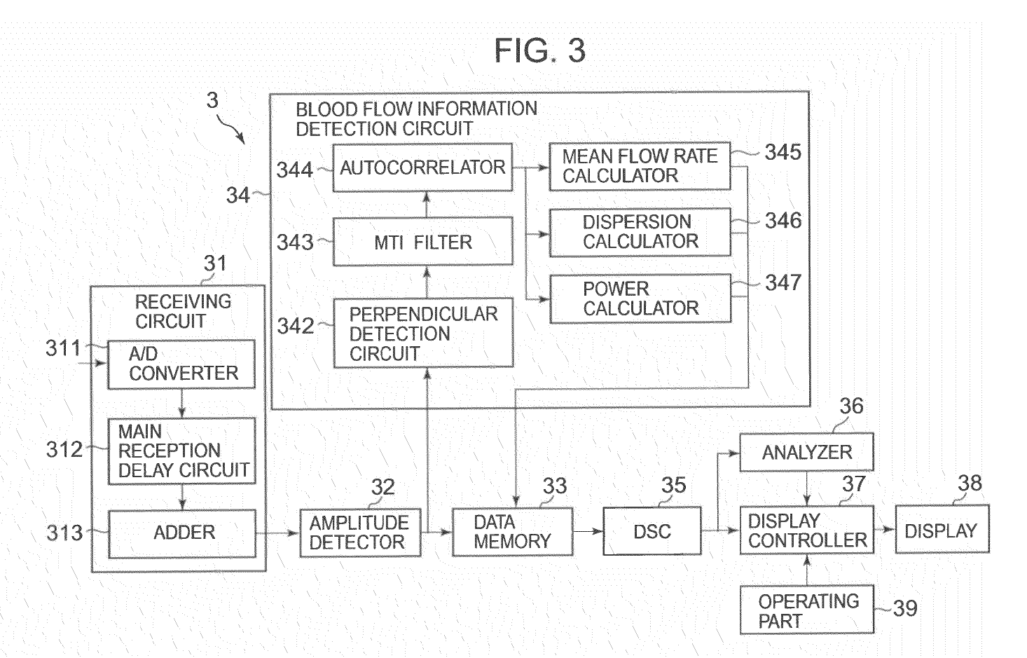

FIG. 3 is a configuration diagram showing the internal configuration of the apparatus body 3. As shown in FIG. 3, the apparatus body 3 has a receiving circuit 31, an amplitude detector 32, a data memory 33, a blood flow information detection circuit 34, a DSC (digital scan converter) 35, an analyzer 36, a display controller 37, a display 38, and an operating part 39.

Echo signals are input into the receiving circuit 31 from the ultrasound probe 2. This receiving circuit 31 has an A/D (analog to digital) converter 311, a main reception delay circuit 312, and an adder 313. The A/D converter 311 converts signals that are input from the ultrasound probe 2 into digital signals. The main reception delay circuit 312 provides a delay time that is required to determine the receiving directionality for the echo signals. The adder 313 generates a signal echo signal by phasing and adding multiple echo signals that have been input.

Echo signals are input into the amplitude detector 32 from the receiving circuit 31. The amplitude detector 32 detects envelopes. The amplitude detector 32 detects the intensity of the received ultrasound by implementing compression with logarithmic conversion on the detected data. The data of that intensity value becomes an element of a B-mode image due to its correspondence with the reflected position of the ultrasound.

The blood flow information detection circuit 34 has a perpendicular detection circuit 342, a MTI (moving target indicator) filter 343, an autocorrelator 344, a mean flow rate calculator 345, a dispersion calculator 346, and a power calculator 347.

The perpendicular detection circuit 342 extracts Doppler signals that have shift frequency components from echo signals, using perpendicular detection processing. The MTI filter 343 implements high-pass filter processing that separates tissue signals and blood flow signals from the Doppler signals. The autocorrelator 344 implements frequency analysis of the Doppler signals to obtain the shift frequency of the blood flow. The mean flow rate calculator 345 calculates the mean velocity of the blood flow from the shift frequency. The dispersion calculator 346 calculates velocity distribution of the blood flow from the shift frequency. The power calculator 347 calculates power that reflects the blood flow volume from the shift frequency.

Data representing the mean average, velocity distribution, and power of the blood flow constitute a Color Doppler mode image, and is sent to the data memory 33.

The data memory 33 stores the data of each pixel value constituting a B-mode image corresponding to the positions where the ultrasound was reflected. In addition, the data memory 33 stores the data representing the mean average, velocity distribution, and power of the blood flow, which constitute a Color Doppler mode image corresponding to the positions where the ultrasound was reflected.

The DSC 35 constructs volume data according to the actual space from signals of B-mode image and Color Doppler mode image stored in the data memory 33. The DSC 35 distributes B-mode image and Color Doppler mode image of each scan frame stored in the data memory 33 in a three-dimensional memory space based on positional information detected by the position detector 24 and the oscillation angle of the scanning line.

The analyzer 36 specifies a fetus based on a three-dimensional image generated by the DSC 35. If there are multiple fetuses present in the three-dimensional image, each fetus is separately specified.

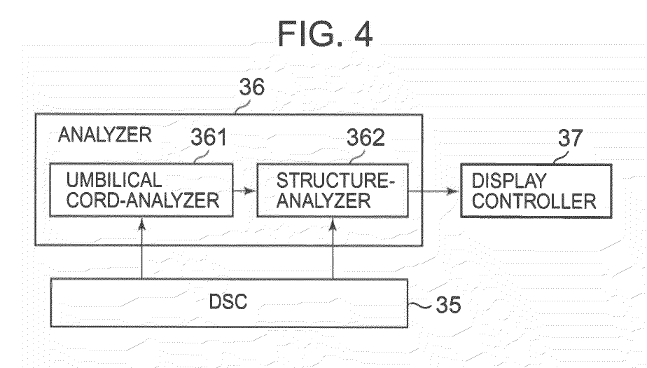

FIG. 4 is a diagram showing the detailed configuration of the analyzer 36 according to the first embodiment. As shown in FIG. 4, the analyzer 36 has an umbilical cord-analyzer 361 that specifies an umbilical cord, and a structure-analyzer 362 that specifies a structure continuing from the umbilical cord. The analyzer 36 first specifies the umbilical cord from a three-dimensional image. Next, the analyzer 36 specifies multiple fetuses respectively by specifying the structures continuing from the umbilical cord. The structure continuing from the umbilical cord is a fetus.

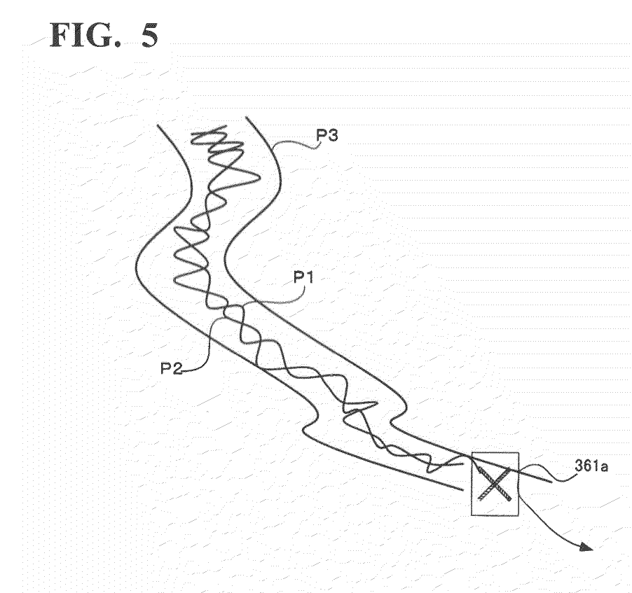

Specifying the umbilical cord is described based on FIG. 5. FIG. 5 is a pattern diagram showing a Color Doppler image of the umbilical cord.

The umbilical cord-analyzer 361 specifies a region occupied by the umbilical cord P3 using the Color Doppler mode image. The umbilical cord P3 has a characteristic pattern in blood vessels running therein. For the blood vessels in the umbilical cord P3, an artery P1 and a vein P2 run in a spiral. So, the umbilical cord-analyzer 361 specifies the region occupied by the umbilical cord P3 by detecting this spiral running pattern of blood vessels.

Specifying the region of the umbilical cord P3 with the umbilical cord-analyzer 361 is specifically as follows. The umbilical cord-analyzer 361 has prestored a predicted running pattern of blood vessels 361a. This predicted running pattern of blood vessels 361a is image data with crossing lines that represent blood vessels. The umbilical cord-analyzer 361 compares the data of each image, for the entire region of the Color Doppler mode image, to this predicted running pattern of blood vessels 361a. The umbilical cord-analyzer 361 makes the comparison while zooming in and out the predicted running pattern of blood vessels 361a according to a predefined rule and varying the crossing angle of the lines. Then, when the image data array pattern of the comparison subject and the predicted running pattern of blood vessels 361a match, a region comprising that image data is specified as a portion of the region occupied by the umbilical cord P3.

The umbilical cord-analyzer 361 scans the entire region of the Color Doppler mode image for all the umbilical cords P3 respectively.

The umbilical cord-analyzer 361 regards as one umbilical cord P3, any region where the region matching the predicted running pattern of blood vessels 361a continues on. To continue on means that matched regions are connected with each other by the same artery P1 and vein P2.

In other words, the umbilical cord-analyzer 361 determines whether any blood vessel is present between neighboring regions matched to the predicted running pattern of blood vessels 361a. If a blood vessel is present, the umbilical cord-analyzer 361 tracks that blood vessel to determine whether the regions to be determined are connected with each other by this blood vessel. Moreover, if the umbilical cord-analyzer 361 determines that the regions are connected with each other, it determines that those regions and the blood vessel region therebetween constitute the umbilical cord P3. Then, the umbilical cord-analyzer 361 puts a series of regions connected by the same blood vessels into one group. On the other hand, other matching regions that have been determined by the umbilical cord-analyzer 361 not to be in a relationship with the series of regions for this group are regarded as constituting another umbilical cord P3. Moreover, the umbilical cord-analyzer 361 further puts a group of regions that have a continuing region relationship with those matching regions into the other group. Grouping by the umbilical cord-analyzer 361 is done by associating with the same fetus ID a collection of positional information that indicates the position of each region that belongs to one group.

In addition, the umbilical cord-analyzer 361 may detect a corded structure using a B-mode image to specify the umbilical cord P3. In this case, the umbilical cord-analyzer 361 detects the corded structure by detecting a collection of portions having diameters below the predefined value in the image.

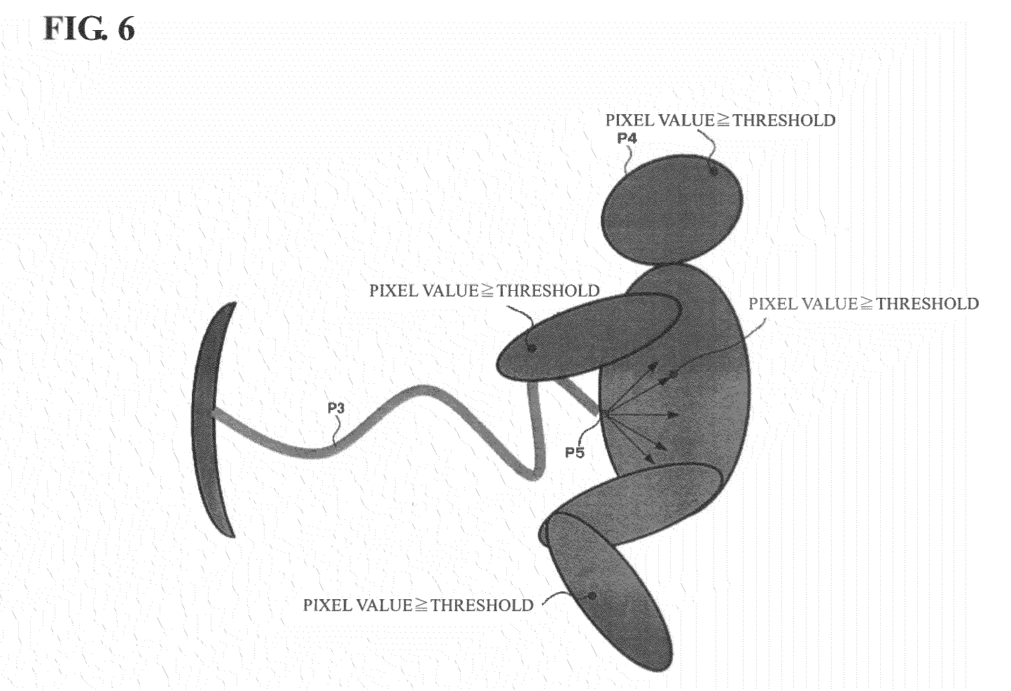

Next, specifying the fetus is described based on FIG. 6. FIG. 6 is a pattern diagram showing a B-mode image of a region including a fetus.

The structure-analyzer 362 specifies a fetus using the B-mode image. This structure-analyzer 362 specifies a collection of voxels continuing into the region of the umbilical cord P3 specified by the umbilical cord-analyzer 361 and having a pixel value above a predefined threshold. The predefined threshold is a value that characterizes brightness exhibited by a structure P4 present in the womb and that is also an empirical value.

Specifying a fetus using the structure-analyzer 362 is specifically performed as follows. The structure-analyzer 362 tracks the region occupied by the specified umbilical cord P3, with a reference point P5 as its end point. This reference point P5 is a connection point between the umbilical cord P3 and the structure P4.

Then, the structure-analyzer 362 expands the voxels of the comparison subject from this reference point P5 and compares the pixel values of the voxels of the comparison subject with the predefined threshold. As a result of comparison by the structure-analyzer 362, if a pixel value is above the predefined threshold, the structure-analyzer 362 regards that voxel of the comparison subject as one component of the structure P4. On the other hand, if the pixel value is below the predefined threshold, the structure-analyzer 362 does not regard that voxel as one component of the structure P4. In addition, the structure-analyzer 362 does not expand the voxels around the comparison subject having pixel values below the predefined threshold.

If there are multiple umbilical cords P3 specified by the umbilical cord-analyzer 361, the structure-analyzer 362 specifies a structure P4 continuing into the umbilical cord for each umbilical cord P3. When the structure P4 is specified by the structure-analyzer 362, the umbilical cord P3 continuing into that structure P4 is regarded as being in the same group as the structure P4. In other words, it further associates a collection of positional information for voxels continuing from one umbilical cord P3 and having a pixel value above the predefined threshold, with the fetus ID associated with that one umbilical cord P3.

In addition, the analyzer 36 may model a collection of associated positional information using models such as rectangular solids and spheres and further associate the type and arranged position of the models and the coupling relationship between the models with the fetus ID.

The display controller 37 receives voxels of all the structures P4 output from the DSC 35, and implements volume rendering processing and color processing on them to constitute one screen. The display controller 37 outputs it to the display 38. The display 38 is a liquid crystal display, an organic EL display, CRT, etc.

In volume rendering processing, the display controller 37 takes a sample of the pixel value of each voxel in all the structures P4 from the direction of the viewpoint, and calculates light transmission according to the opacity and reflection with respect to the viewpoint, and applies shading, thereby generating a projected image. The viewpoint in volume rendering processing is input by an operator via an operating part 39. The operating part 39 is a keyboard, a mouse, a trackball, etc. When the direction of the viewpoint, angle around the axis of the viewpoint, viewpoint, and distance to the three-dimensional image are input by physicians and others via the operating part 39, the display controller 37 sets the viewpoint corresponding to that operation, i.e., the direction of the viewpoint, rotational angle of the image, and zoom-in volume, so as to implement rendering processing on the three-dimensional image.

In color processing, a hue, such as one in the RGB format, is assigned to each voxel of the structures P4. This hue that is assigned is different for each structure P4.

This color processing is specifically performed as follows. The display controller 37 obtains a collection of positional information associated with one fetus ID from the analyzer 36. The display controller 37 also assigns the same hue to each voxel specified based on that obtained positional information. Similarly, the display controller 37 obtains a collection of positional information for other fetus IDs and assigns the same hue for each collection of the positional information based on the obtained positional information. The display controller 37 assigns a different hue to a group with a different fetus ID. For example, the ultrasound imaging apparatus 1 has prestored a list of corresponding fetus IDs and hues. The analyzer 36 assigns a fetus ID to a group from the list. Then, the display controller 37 refers to the list and assigns a hue corresponding to the fetus ID.

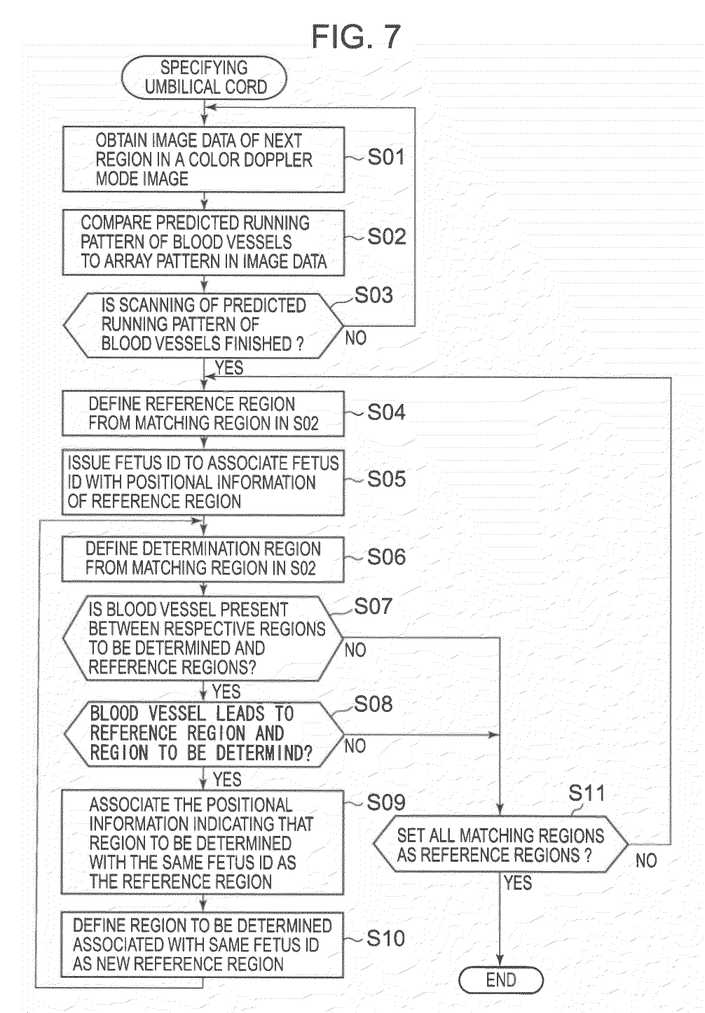

FIGS. 7 through 9 are flowcharts showing operation of the ultrasound imaging apparatus 1 to identify and display the fetus.

First, FIG. 7 is a flowchart showing the process of specifying the umbilical cord P3. The umbilical cord-analyzer 361 obtains image data of the next region in a Color Doppler mode image (S01). When the image data is obtained, the umbilical cord-analyzer 361 compares the predicted running pattern of blood vessels 361a to the array pattern in that image data (S02). The umbilical cord-analyzer 361 implements S01 and S02 until all the regions are scanned (S03, No).

When scanning using the predicted running pattern of blood vessels 361a is finished (S03, Yes), among matching regions matched by comparison at S02, the umbilical cord-analyzer 361 defines as a reference region any matching region that is located in the outermost outline on the Color Doppler mode image and has not yet become a reference region (S04). Moreover, the umbilical cord-analyzer 361 issues a new fetus ID to associate the fetus ID with the positional information of that reference region (S05).

When the reference region is determined, the umbilical cord-analyzer 361 searches for each matching region present within a predefined search range from this reference region, which is defined in advance. As a result of the search, the umbilical cord-analyzer 361 defines regions other than the reference regions as continuous regions to be determined (S06).

Then, the umbilical cord-analyzer 361 determines whether any blood vessel is present between any of respective regions to be determined and the reference regions (S07). When there is a blood vessel (S07, Yes), the umbilical cord-analyzer 361 tracks that blood vessel to determine whether it leads to a reference region and a region to be determined (S08). If it does (S08, Yes), the umbilical cord-analyzer 361 associates the positional information indicating that region to be determined with the same fetus ID as the reference region (S09).

Moreover, the umbilical cord-analyzer 361 defines the region to be determined associated with the same fetus ID as a new reference region (S10) and repeats S06-S09.

As a result of the determination by the umbilical cord-analyzer 361, there may be no regions to be determined where blood vessels are present between those regions and a reference region (S07, No) and also there may be no regions to be determined that are connected with a reference region via a blood vessel (S08, No), and moreover, there may be the case where all the matching regions have not been set as reference regions (S11, No). In these cases, the umbilical cord-analyzer 361 repeats processing from S04 to S10. In other words, the umbilical cord-analyzer 361 issues a new fetus ID for a matching region not associated with a fetus ID and searches for a new umbilical cord P3 that includes that region.

If the umbilical cord-analyzer 361 has set all the matching regions as reference regions (S11, Yes), the process of specifying the umbilical cord P3 is finished.

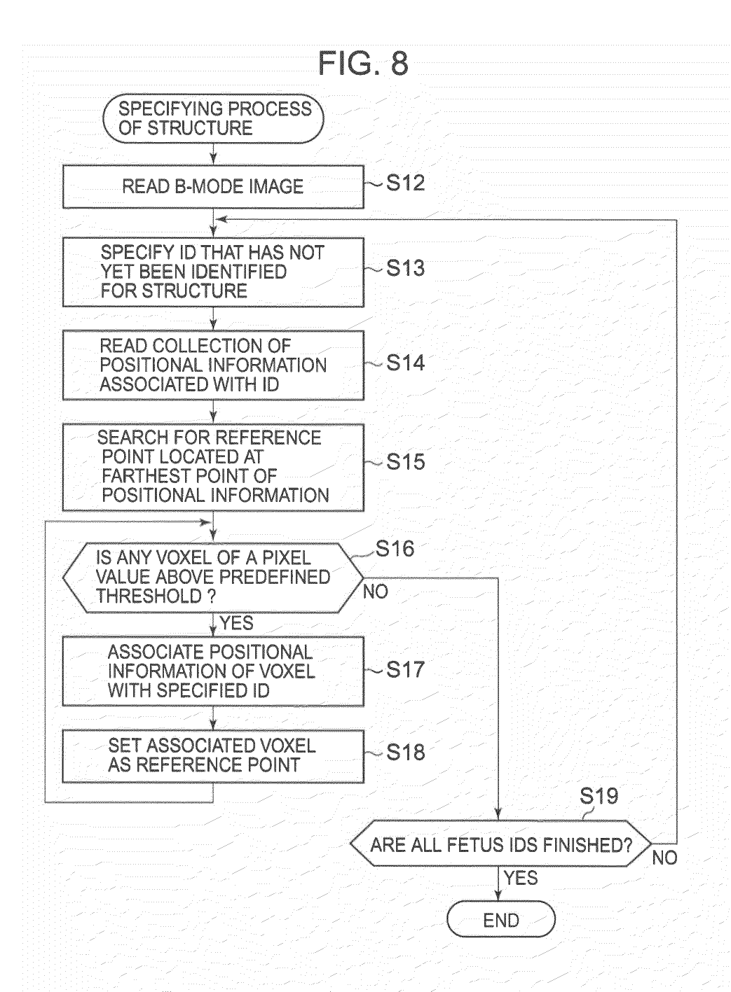

Next, FIG. 8 is a flowchart showing the process of specifying the structure P4. When the process of specifying the umbilical cord P3 using the umbilical cord-analyzer 361 is finished, the structure-analyzer 362 reads a B-mode image (S12). Then, the structure-analyzer 362 specifies a fetus ID that has not yet been identified for the structure P4 (S13). Moreover, the structure-analyzer 362 reads the collection of positional information associated with that fetus ID (S14).

When the structure-analyzer 362 reads the B-mode image and collection of positional information, the structure-analyzer 362 further searches for a reference point P5 located at the farthest point of that positional information (S15). Then, the structure-analyzer 362, using as comparison subjects, from among the voxels around that reference point P5, voxels that have not yet been determined to be one component of the structure P4, compares the predefined threshold to the pixel value of those voxels. (S16).

As a result of the comparison, when there is any voxel that has a pixel value above the predefined threshold (S16, Yes), the structure-analyzer 362, treating that voxel as one component of the structure P4, associates positional information of that voxel with the specified fetus ID (S17).

Moreover, the structure-analyzer 362 handles each voxel that has been made one component of the structure P4 as a reference point P5 (S18), and repeats S16 and S17. On the other hand, as a result of the comparison, if there is no voxel that has a pixel value above the predefined threshold (S16, No) and if specifying all the fetus IDs is not finished (S19, No), structure-analyzer 362 repeats S13-S19. In this way, it implements the process of specifying the structure P4 for a new fetus ID.

If specifying the structures P4 for all the fetus IDs is finished (S19, Yes), the structure-analyzer 362 finishes the process of specifying the structure P4 continuing into the umbilical cord for each fetus ID, in other words, for each specified umbilical cord P3.

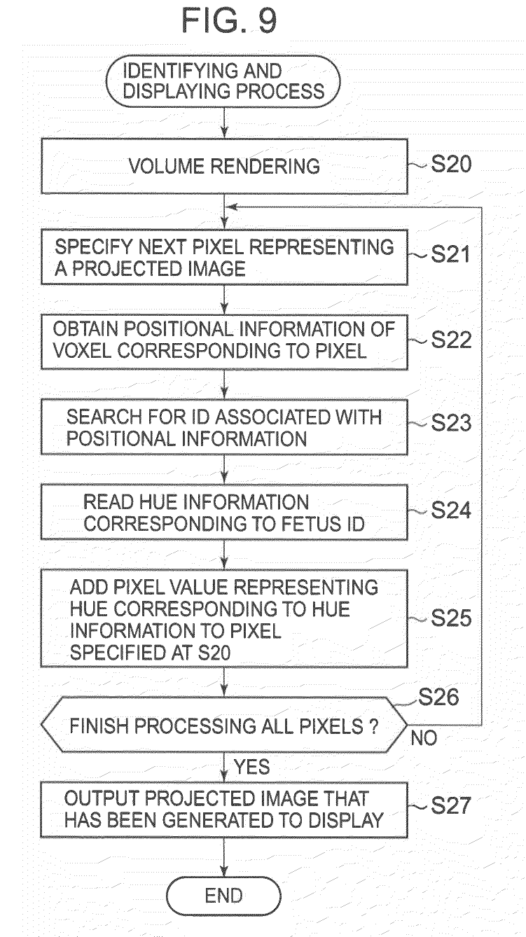

Next, FIG. 9 is a flowchart showing the process of identifying the structure P4. When the process of specifying the structure P4 is finished, from the viewpoint set by the operator via the operating part 39, the display controller 37 implements the process of volume rendering on the collection of voxels indicated by the positional information associated with all the fetus IDs (S20).

Moreover, the display controller 37 specifies one pixel representing a projected image generated by the volume rendering process (S21). The display controller 37 obtains positional information of the voxel corresponding to the specified pixel (S22). When the display controller 37 obtains positional information, it searches for a fetus ID associated with the obtained positional information (S23). The display controller 37 refers to the list and reads hue information corresponding to the fetus ID (S24).

When the display controller 37 reads hue information, it adds a pixel value representing the hue corresponding to that hue information to the pixel specified at S20 (S25).

The display controller 37 repeats processing from S21 to S25 for all the pixels representing the projected image (S26, No). When the display controller 37 finishes processing all the pixels (S26, Yes), the display controller 37 outputs the projected image that has been generated to the display 38 (S27).

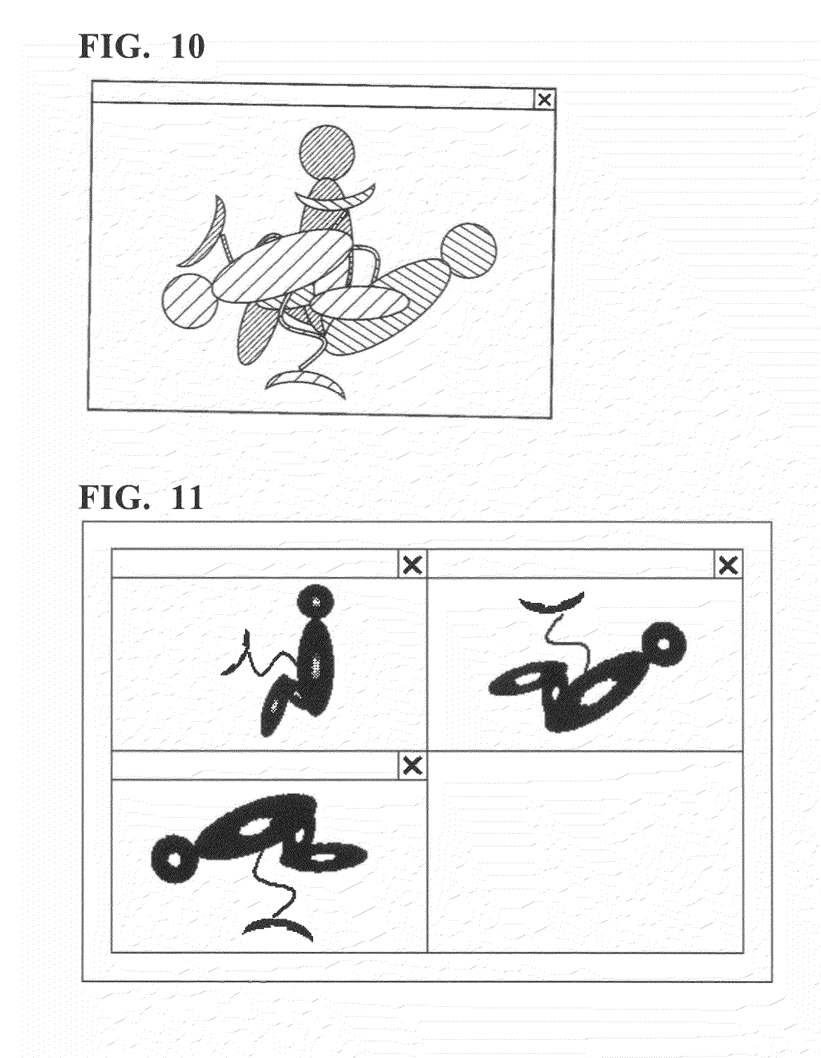

FIG. 10 is a pattern diagram showing a screen from the ultrasound imaging apparatus 1. As shown in FIG. 10, the structures P4, i.e., the fetuses, are displayed on the screen, with each individual being distinguished by color. Therefore, even when there are multiple fetuses in the womb, it is easy for physicians and others to confirm the condition of each fetus, thus improving the accuracy of measuring the growth condition of the fetuses, etc.

FIG. 11 is a pattern diagram showing a second aspect of a screen from the ultrasound imaging apparatus 1. Although the case where the fetuses are displayed such that they are distinguished by color has been described above, alternatively, as shown in FIG. 11, the screen may be divided so as to display a separate window for each fetus.

In this case, from the viewpoint set by the operator via the operating part 39, the display controller 37 implements a volume rendering process for each collection of voxels indicated by positional information associated with each fetus ID. Then, the display controller 37 causes the respective projected images to be displayed in separate windows.

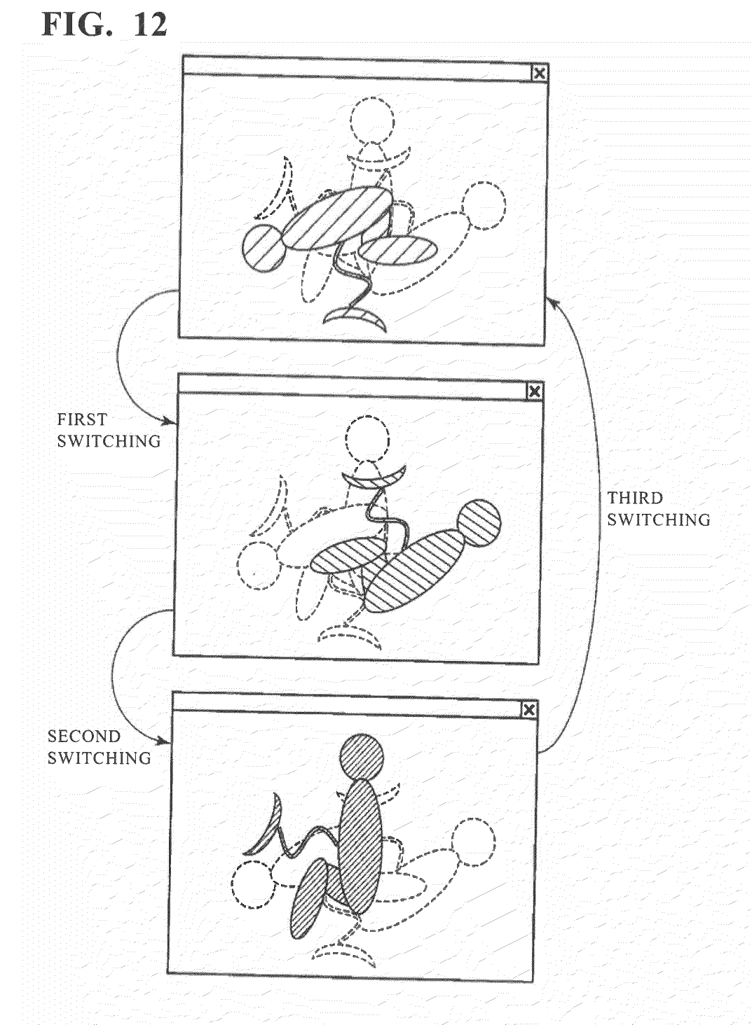

FIG. 12 is a pattern diagram showing a third aspect of a screen from the ultrasound imaging apparatus 1. Although the examples of identification and display by means of distinguishing fetuses by color and dividing the screen for each fetus have been described above, alternatively, as shown in FIG. 12, the display controller 37 may display only one fetus in response to a switching operation via the operating part 39.

In this case, in the volume rendering process at S20, from the viewpoint set by the operator via the operating part 39, the display controller 37 implements a volume rendering process per collection of voxels indicated by positional information associated with each fetus ID. Then, the display controller 37 switches each P4 that has undergone volume rendering to cause it to be displayed in series, according to the number of times the switching operation has taken place.

For example, suppose that there is a collection of positional information associated with fetus IDs A, B, and C respectively, i.e. there are three fetuses. In the first display, the display controller 37 implements a volume rendering process on the collection of voxels indicated by positional information associated with the fetus ID A. The display controller 37 causes only the fetus obtained by the process to be displayed.

When the first switching operation is performed, the display controller 37 implements a volume rendering process on the collection of voxels indicated by positional information associated with the fetus ID B. The display controller 37 causes only the fetus obtained by the process to be displayed.

When the second switching operation is performed, the display controller 37 implements a volume rendering process on the collection of voxels indicated by positional information associated with the fetus ID C. The display controller 37 causes only the fetus obtained by the process to be displayed.

When the third switching operation is performed, returning to the first display, the display controller 37 implements a volume rendering process on the collection of voxels indicated by positional information associated with the fetus ID A The display controller 37 causes only the fetus obtained by the process to be displayed.

With these modes of identification and display indicated by FIGS. 11 and 12 also, it becomes easy for physicians and others to identify multiple fetuses, improving the accuracy of measuring the growth condition of the fetuses, etc.

Next, the second embodiment for identifying and displaying fetuses is described. The ultrasound imaging apparatus 1 according to the second embodiment specifies the same fetuses both in the past and in new examinations. More particularly, specification using this ultrasound imaging apparatus 1 is as follows. The ultrasound imaging apparatus 1 stores the position of the placenta connected to a fetus.

The ultrasound imaging apparatus 1 specifies the same fetus in a new examination based on the stored placenta position. Because the fetus moves about freely in the womb and the umbilical cord also moves following it, it is difficult for physicians and others to specify the same fetus based on the position where the fetus is present, etc.

However, because the placenta position is specific to each fetus and that position has been determined, by linking the placenta position with the fetus connected to it, the same fetus can be identified.

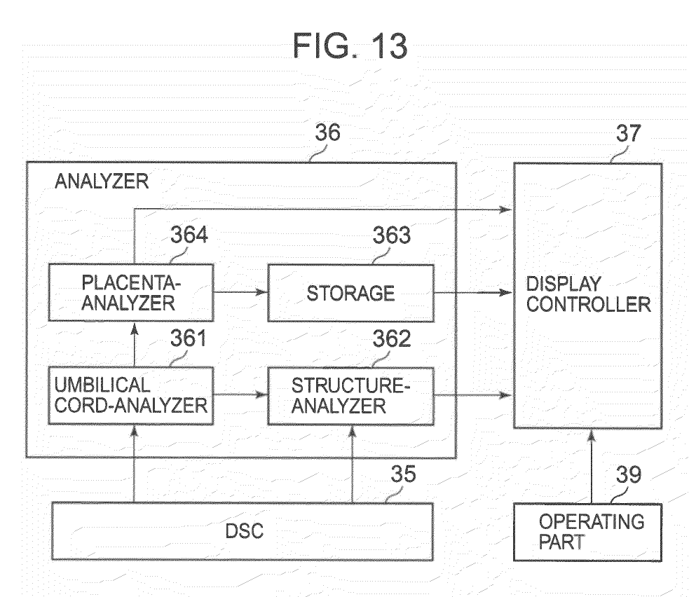

FIG. 13 is a configuration diagram of the analyzer 36 according to the second embodiment. As shown in FIG. 13, in addition to the umbilical cord-analyzer 361 and the structure-analyzer 362, this analyzer 36 has a storage 363 and a placenta-analyzer 364.

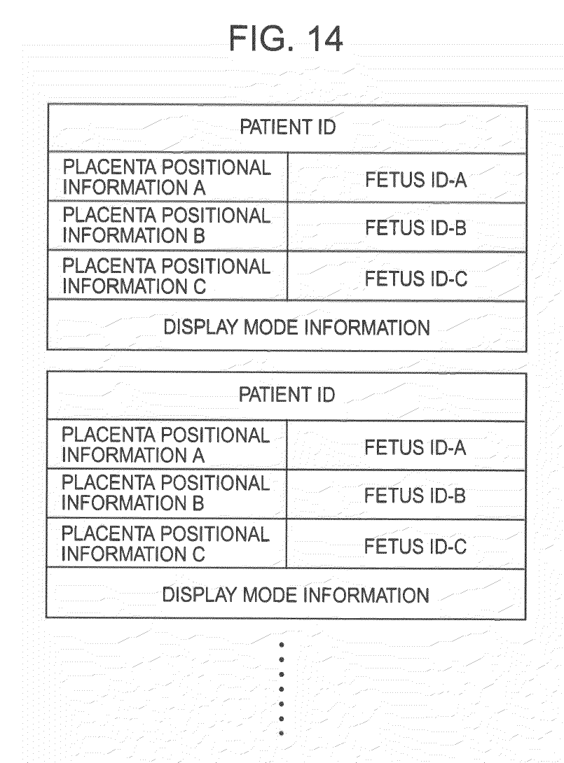

The storage 363 is nonvolatile memory. As shown in FIG. 14, for each patient ID, the storage 363 stores combinations of fetus IDs and placenta positional information, and display mode information. The placenta positional information is information indicating the position at the farthest point opposite the structure P4 of the umbilical cord P3, which is represented using coordinates (X, Y, Z). The display mode information is indicated by the position of the viewpoint, rotational angle and distance to the three-dimensional image, which are set by the operator via the operating part 39. The patient ID is the ID of a subject, which is preinput by the operator via the operating part 39. In other words, the storage 363 stores placenta positional information for each structure P4 that continues into the same umbilical cord as that placenta; that is, for each fetus ID.

The placenta-analyzer 364, in each examination, specifies the position of the placenta that continues into each umbilical cord P3 and generates placenta positional information based on the specified position. More particularly, it tracks the collection of positional information obtained by the umbilical cord-analyzer 361 and defines the positional information as the farthest position of the placenta. The placenta-analyzer 364, from both ends of the umbilical cord P3, defines as the placenta positional information an end point that is opposite to the end point defined as reference point P5 by the structure-analyzer 362.

In addition, the placenta-analyzer 364 causes the placenta positional information to be stored in the storage 363. More particularly, the placenta-analyzer 364 obtains a fetus ID associated with the collection of positional information that indicates the tracked umbilical cord P3. The placenta-analyzer 364 associates this obtained fetus ID with the placenta positional information to cause it to be stored under the patient ID.

If the combination of fetus ID and placenta positional information has been already stored for the patient ID, in order to incorporate changes in the placenta position depending on differences in gestational weeks so as to improve the accuracy, the placenta-analyzer 364 may replace the combination with the new combination, thereby causing it to be stored. It is desirable that the replacing and storing is implemented after the placenta-analyzer 364 reproduces the identifying mode of the fetus used in the previous examination, e.g., after the examination is finished. Alternatively, first-time only storage of the placenta positional information may be implemented.

In a new examination, if the patient ID input using the operating part 39 has been stored in the storage 363, the display controller 37 adds a hue that corresponds to the fetus ID in the list, combined with the placenta positional information generated at the placenta-analyzer 364, to the structure P4 that continues from the placenta position indicated by the placenta positional information generated by this placenta-analyzer 364, and causes it to be displayed.

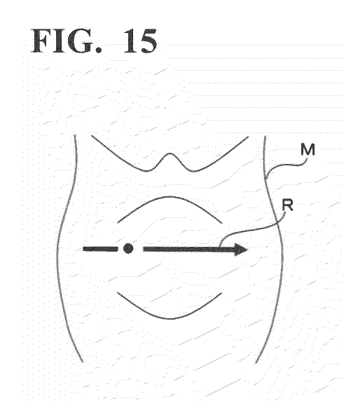

In comparing the placenta positional information obtained in a new examination to the placenta positional information stored in the storage 363, it is necessary that these sets of placenta positional information be represented with the same coordinate system. Therefore, before starting the examination, the display controller 37 causes a probe movement route R that guides the movement route of the ultrasound probe 2 to be displayed on the display 38, as shown in FIG. 15. The probe movement route R is the start position of scanning with the ultrasound probe 2; in other words, an arrow indicating the origin position at which the ultrasound probe 2 is initially placed, and the direction in which the ultrasound probe 2 is to be moved.

The display controller 37 prestores a subject model M, origin position, and directional information for drawing this probe movement route R. The display controller 37 draws the probe movement route R pointing in the direction indicated by the directional information from the origin point, and causes it to be displayed on the display 38.

In addition, if the patient ID input using the operating part 39 has been stored in the storage 363, the display controller 37 implements a volume rendering process at the viewpoint indicated by the display mode information stored in association with that patient ID. This display mode information is stored in association with the patient ID by the display controller 37.

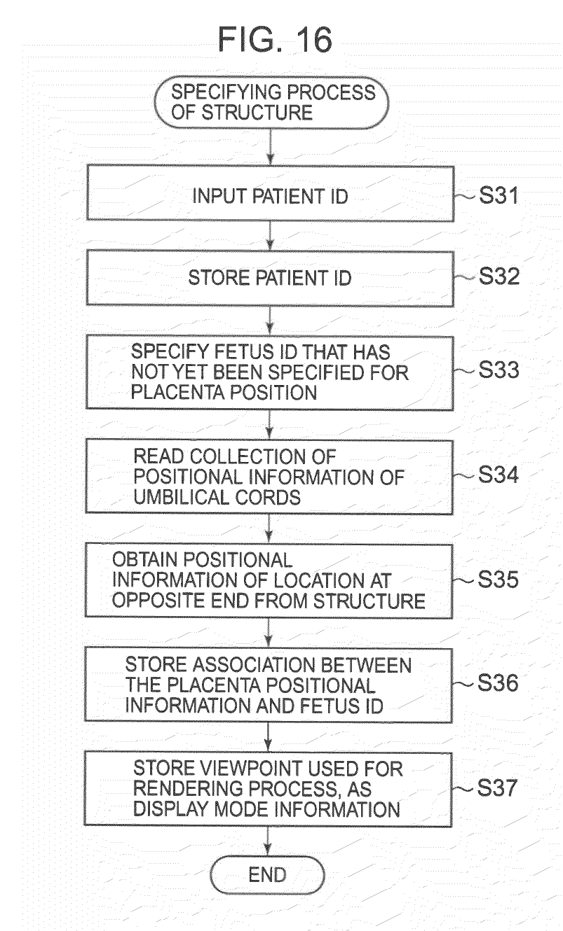

FIG. 16 is a flowchart showing an operation to store the placenta position in the storage 363. First, the patient ID is input in advance by the operator via the operating part 39 (S31). If the input patient ID has not been stored in the storage 363, the placenta-analyzer 364 causes that patient ID to be stored (S32).

Subsequently, after the umbilical cord-analyzer 361 specifies the umbilical cord P3, the structure-analyzer 362 specifies the structure P4.

Either after the structure P4 is specified or after an image is output, the placenta-analyzer 364 first specifies a fetus ID that has not yet been specified for the placenta position (S33). The placenta-analyzer 364 reads an umbilical cord P3 part based on the collection of positional information associated with the specified fetus ID (S34). When the umbilical cord P3 is read, the placenta-analyzer 364, from among the positional information of the umbilical cord P3, obtains positional information of the location at the opposite end from the structure P4 (S35).

When the positional information is obtained, the placenta-analyzer 364 links the association between the placenta positional information represented by this positional information and the fetus ID specified at S31 with the input patient ID, and causes it to be stored in the storage 363 (S36).

Next, triggered by end of the examination, etc., the display controller 37 links the viewpoint used for the volume rendering process, as the display mode information, with the input patient ID, and causes it to be stored in the storage 363 (S37).

In this way, for the past examinations of the subject represented with the patient ID, the fact that structure P4 specified with the placenta positional information has been displayed in the identifying mode is stored, represented by the fetus ID, in addition to which the viewpoint at that time is stored.

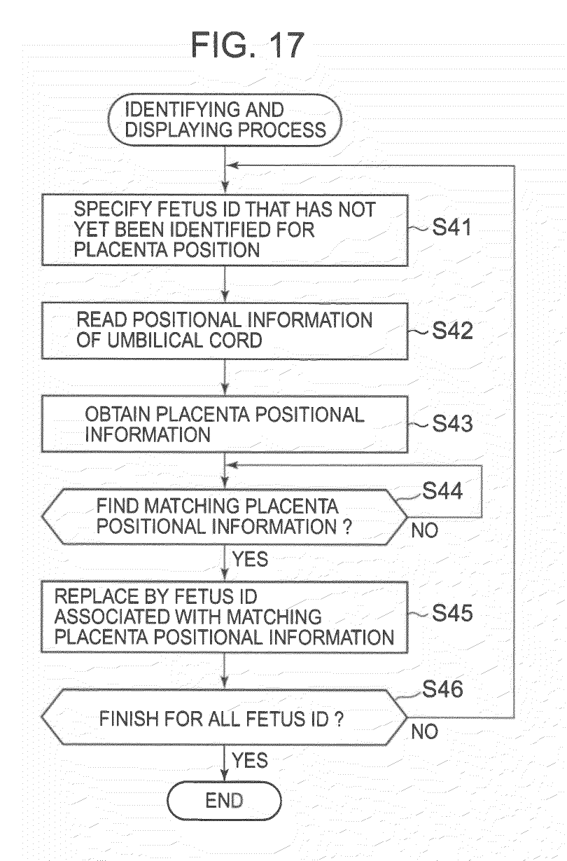

FIG. 17 is a flowchart showing an operation to reproduce the mode of identifying the fetus, even in a new examination.

First, among fetus IDs issued at S05 in the current examination, the placenta-analyzer 364 specifies a fetus ID that has not yet been identified for the placenta position (S41). The placenta-analyzer 364 reads an umbilical cord P3 part from the collection of positional information associated with that fetus ID (S42). When the umbilical cord P3 is read, the placenta-analyzer 364, from among the positional information of the umbilical cord P3, obtains positional information of a location at the opposite end from the structure P4, as placenta positional information (S43).

When the placenta positional information is obtained, the display controller 37 compares the obtained placenta positional information to each placenta positional information associated with the preinput patient ID (S44). When the comparison finds matching placenta positional information (S44, Yes), the display controller 37 replaces the fetus ID specified at S41 by the fetus ID associated with that placenta positional information (S45).

In addition, in the comparison of placenta positional information, if a coordinate (x, y, z) indicated by the obtained placenta positional information is within a sphere of a predefined diameter around the coordinate (X, Y, Z) indicated by the placenta positional information stored in the storage 363, both sets of information are considered to be a match.

In the current examination, if processing is not finished for all fetus IDs issued at S05 (S46, No), S41-S46 are repeated for the next fetus ID. Then, if processing is finished for all fetus IDs (S46, Yes), the display controller 37 implements the processes of identifying and displaying, starting from S20.

In addition, the display controller 37 may store the origin position of the probe movement route R for each gestational week, and cause the probe movement route R, extending from the origin point according to the gestational weeks input via the operating part 39, to be displayed.

Also, the ultrasound imaging apparatus 1 may store the route along which the ultrasound probe 2 was actually moved, and convert the coordinates of placenta positional information obtained in a new examination from differences in the movement route of the new examination.

Specifically, the display controller 37 causes the subject model M to be displayed on the display 38 after the examination. Then, when the actual route along which the ultrasound probe 2 was moved in the examination is input by the operator via the operating part 39, the display controller 37 causes that route to be drawn on the subject model M and also associates the origin position and directional information indicated by that route with the patient ID, and causes it to be stored in the storage 363.

Upon a new examination, the display controller 37 generates a determinant that converts the coordinate system of the route input using the operating part 39 into the coordinate system indicated by the route stored in the storage 363. The display controller 37 converts the coordinates indicated by placenta positional information obtained in the new examination using this determinant.

Then, the display controller 37 compares this placenta positional information after conversion to each placenta positional information stored in the storage 363 and colors the structure P4 with a hue corresponding to the fetus ID associated with matching placenta positional information.

In this way, because the ultrasound imaging apparatus 1 according to the second embodiment specifies the same fetus in both past and new examinations, it is possible to reproduce, even in a new examination, the mode of identifying each fetus identified and displayed in past examinations. Therefore, it is possible for physicians and others to eliminate the task of confirming whom an identified and displayed fetus is, which provides good operational efficiency.

What is claimed is:

1. An ultrasound imaging apparatus having a probe configured to transmit ultrasound and receive reflected waves, a generator configured to generate an image based on said reflected waves, and a display that displays said image, wherein the ultrasound imaging apparatus comprising:
a first analyzer configured to specify respectively each umbilical cord present in said image;
a second analyzer configured to respectively specify a structure continuing into one end of said umbilical cord; and
a display controller configure to cause each said structure to be displayed on said identified and display.

2. The ultrasound imaging apparatus according to claim 1, further comprising:
a third analyzer configured to specify a placenta position continuing into the other end of said umbilical cord; and
a storage configured to store said placenta position at the time of a past examination, for each said structure continuing into the same umbilical cord;
wherein said display controller is configured to compare said placenta position at the time of a new examination to the placenta position stored in said storage and to display said structure continuing from substantially the same placenta position as the said past examination, in the same identification mode.

3. The ultrasound imaging apparatus according to claim 2, wherein:
said third analyzer is configured to specify the other end of said umbilical cord as the placenta position.

4. The ultrasound imaging apparatus according to claim 2, further comprising:
an operating part configured to enter an actual route along which said probe moves, upon transmitting and receiving said ultrasound, wherein:
said storage is further configured to store said actual route when storing said placenta; and
said display controller is configured to perform said comparison after transforming said placenta position specified by said third analyzer upon the new examination, based on the coordinate system of said stored actual route.

5. The ultrasound imaging apparatus according to claim 2, wherein:
said display controller is configured to cause said display to display in advance a probe movement route that guides the movement route of said probe before transmitting and receiving the ultrasound.

6. The ultrasound imaging apparatus according to claim 5, wherein:
said display controller is configured to:
store gestational weeks in association with a correction value for correcting said probe movement route; and
cause said probe movement route corrected with the correction value corresponding to the gestational weeks of the subject to be displayed.

7. The ultrasound imaging apparatus according to claim 1, wherein:
said first analyzer is configured to:
store in advance a predicted running pattern of blood vessels running in the said umbilical cord; and
scan said image using the predicted running pattern to identify said umbilical cord.

8. The ultrasound imaging apparatus according to claim 1, wherein:
said display controller is configured to color said each structure with a different color, and cause it to be displayed.

9. The ultrasound imaging apparatus according to claim 1, further comprising:
an operating part configured to enter a switching operation,
wherein said display controller is configured to color only one structure according to said switching operation, and cause it to be displayed.

10. The ultrasound imaging apparatus according to claim 1, wherein:
said display controller is configured to divide a screen for each said structure, and cause it to be displayed.

11. The ultrasound imaging apparatus according to claim 1, further comprising:
an operating part configured to enter a display mode including at least the angle of the image, wherein:
said storage is further configured to store the display mode displayed on said display; and
said display controller is configured to display the image in the same display mode each time.
